(19) United States
(12) Patent Application Publication
(10) Pub. No.: US 2026/0224236 A1
(43) Pub. Date: Aug. 6, 2026

(54) MEDICAL DEVICES, SYSTEMS AND METHODS FOR DELIVERING ENERGY

(71) Applicant: VICORA, INC., Buffalo, NY (US)

(72) Inventors: Richard DUCHARME, Buffalo, NY (US); Daniel RATHBONE, Buffalo, NY (US); Erin GARCIA, Buffalo, NY (US)

(21) Appl. No.: 19/636,540

(22) Filed: Apr. 1, 2026

Related U.S. Application Data
(63) Continuation of application No. PCT/US2024/049656, filed on Oct. 2, 2024.
(63) Continuation-in-part of application No. 18/638,401, filed on Apr. 17, 2024.
(60) Provisional application No. 63/459,955, filed on Apr. 17, 2023.
(60) Provisional application No. 63/587,168, filed on Oct. 2, 2023.

(51) Int. Cl. A61B 17/22; A61B 17/00; A61M 25/00

(52) U.S. Cl. CPC A61B 17/22012; A61M 25/0045; A61B 2017/00017; A61B 2017/00477; A61B 2017/00544; A61B 2017/22051; A61B 2017/22079; A61B 2217/005; A61M 2025/0057; A61M 2202/0007; A61M 2205/0238; A61M 2205/0283

(57) ABSTRACT

A catheter device for the delivery of one or more therapeutic agents comprising: a proximal end portion coupled to a controller configured to provide electrical signals; and a distal end portion comprising one or more electroactive polymer actuators configured for vibrational motion in response to the electrical signals; wherein the catheter device is configured to carry the electrical signals from the controller to the one or more electroactive polymer actuators; and wherein the catheter device is further configured to release one or more therapeutic agents from the distal end portion of the catheter device. The present disclosure also relates to a catheter device comprising a sleeve positioned over the electroactive polymer actuators. The present disclosure further relates to methods and procedures using devices and systems comprising electroactive polymer actuators.

The present application claims priority as a continuation of PCT Application No. PCT/US2024/049656, filed Oct. 2, 2024, which in turn claims priority to U.S. Provisional Application 63/587,168, filed on Oct. 2, 2023. The present application also claims priority as a continuation-in-part of U.S. patent application Ser. No. 18/638,401, filed Apr. 17, 2024, which in turn claims priority to U.S. Provisional Application Ser. No. 63/459,955, filed Apr. 17, 2023. Each of these applications is incorporated herein by reference in their entireties.

### FIELD

The present invention relates to devices enabled by electroactive polymers (“EAP”, e.g., piezoelectric polymers), and more particularly to medical or surgical devices comprising electroactive polymer actuators for delivering energy to a target site. The present invention also relates to systems comprising said devices and to methods using said devices and systems.

### BACKGROUND

Electroactive polymers (“EAP”) are materials with the ability to undergo shape and/or dimensional change in response to electrical stimulation or to convert electrical charges into mechanical or vibrational motion. These properties have been valuable in the medical field, and in particular in the development of medical devices.

Earlier applications include catheters and guidewires, the configuration of which is modifiable by means of EAP actuators, thereby facilitating guiding and navigation through a patient's vasculature.

In U.S. patent application Ser. No. 17/510,194 (published as US 2022/0125454 A1) the inventors describe thrombectomy devices enabled by means of EAP actuators. The contents and disclosure of any publication mentioned in the present application are incorporated herein by reference in their entireties. The thrombectomy devices comprise EAP actuators, which are configured for vibrational motion in response to electrical signals provided by a controller. The electroactive polymer enables the vibration of the distal end of the catheter and breaking up of an occlusion, without transferring motion over substantially the entire length of the catheter. Consequently, the catheter is capable to accessing narrow blood vessels in deeper locations of the vascular space using a long flexible catheter without loss of vibration.

It is an object of the invention to provide improved alternatives to existing devices and systems. It is another object of the invention to provide devices and systems for improved treatments, methods and/or procedures. It is another object of the invention to provide devices and systems for alternative treatments, methods and/or procedures.

### SUMMARY

According to an aspect of the invention, there is provided a catheter device for the delivery of one or more therapeutic agents comprising: a proximal end portion coupled to a controller configured to provide electrical signals; and a distal end portion comprising one or more electroactive polymer actuators configured for vibrational motion in response to the electrical signals; wherein the catheter device is configured to carry the electrical signals from the controller to the one or more electroactive polymer actuators; and wherein the catheter device is further configured to release one or more therapeutic agents from the distal end portion of the catheter device.

In embodiments, the one or more therapeutic agents are released through the catheter device, for example through infusion ports of the catheter device. In embodiments, the distal end portion of the device comprises one or more infusion ports. In embodiments, infusion ports are arranged radially around a circumference of the catheter. In embodiments, infusion ports are arranged longitudinally along the catheter. In embodiments, infusion ports are arranged substantially helically around the catheter.

In embodiments, the catheter device comprises at least one infusion lumen configured to carry the one or more therapeutic agents to the one or more infusion ports. In embodiments, the at least one infusion lumen extends from a proximal end, or proximal end portion of the catheter device, to the one or more infusion ports. In embodiments, the at least one infusion lumen is arranged on the outer surface of the catheter wall, embedded in the catheter wall, or on the inner surface of the catheter wall.

In embodiments, the catheter device comprises two or more infusion lumens, each configured to carry the same therapeutic agent or different therapeutic agents. In embodiments, the catheter device may be configured to deliver two (or more) therapeutic agents which react when they come into contact with each other.

In embodiments, the catheter device, preferably the proximal end of the catheter device, is coupled to a pumping or aspiration means. In embodiments, the catheter comprises at least one aspiration lumen configured to carry one or more occlusions or occlusion fragments from the distal end or distal end portion of the catheter device to the proximal end of the catheter device. In embodiments, the lumens (e.g., the infusion lumen and the aspiration lumen, and any other device lumen mentioned in the present disclosure) are concentrically arranged. In embodiments, the lumens are arranged side-by-side and/or around the longitudinal axis of the catheter body.

In embodiments, the catheter device is configured to release the one or more therapeutic agents from the distal end or the distal opening, of the catheter body. The catheter device may be configured to release one or more therapeutic agents carried through one or more infusion lumens from the distal openings of the lumen.

In embodiments, the catheter device is configured to selectively open or close the distal end of the catheter body, the distal opening of the catheter body and/or the distal opening of the infusion lumen(s). In embodiments, the catheter device comprises one or more EAP actuators configured to modify the configuration of the distal end of the catheter body, the distal opening of the catheter body and/or the distal opening of the infusion lumen(s), in response to electrical signals. In embodiments, the catheter device comprises one or more EAP actuators configured to selectively open or close the distal end of the catheter body, the distal opening of the catheter body and/or the distal opening of the infusion lumen(s), in response to electrical signals. For example, such EAP actuators are configured to translate electrical signals into a first expanded configuration in which the therapeutic agent(s) may be released and/or to a second contracted configuration in which the therapeutic agent(s) may not be released.

The therapeutic agent may be any therapeutic agent for the local or systemic treatment of a patient. The therapeutic agent may be any therapeutic agent used in connection to the methods, treatments and procedures described in the present application. In embodiments, the therapeutic agent is a lytic agent, such as a lytic enzyme (e.g., alteplase), or an embolic agent (e.g., glue).

In embodiments, the therapeutic agent is comprised in microbubbles. In embodiments, the microbubbles comprise a core including one or more therapeutic agents and a shell encapsulating said core. The core may, alternatively or additionally comprises a liquid or gas.

In embodiments, the shell comprises or consists of surfactants, proteins, lipids and/or polymers, including denatured proteins, biocompatible polymers, phospholipids, and a combination thereof.

In embodiments, the one or more therapeutic agents may be stored in a therapeutic agent source (e.g., a syringe, bottle, cartridge, or bag), which may be fluidly coupled to the proximal end of the catheter device or to the proximal end of a system comprising the catheter device.

In embodiments, the catheter device comprises at least one occlusion device. The occlusion device may be integrated with or coupled to the catheter device. Alternatively, the catheter device according to the present disclosure may be provided, as a system or kit, with a separate occlusion device. In embodiments, the occlusion device may comprise an expandable structure, such as an expandable balloon.

In embodiments, the expandable structure is an inflatable structure. In embodiments, the catheter device or the occlusion device comprises at least one inflation lumen in fluid communication with a cavity of the inflatable structure. In embodiments, the at least one inflation lumen extends from a proximal end, or proximal end portion of the catheter device, to the inflatable structure. In embodiments, the at least one inflation lumen is arranged on the outer surface of the catheter wall, embedded in the catheter wall, or on the inner surface of the catheter wall. In embodiments, the lumens (e.g., the infusion lumen, the aspiration lumen, the inflation lumen, and any other device lumen mentioned in the present disclosure) are concentrically arranged. In embodiments, the lumens are arranged side-by-side and/or around the longitudinal axis of the catheter body.

In embodiments, the occlusion device comprises one or more electroactive polymer actuators configured to expand and/or contract the expandable structure in response to electrical signals. In embodiments, one or more electroactive polymer actuators are located on an inner surface of the expandable structure, on an outer surface of the expandable structure, and/or are embedded in the expandable structure. In embodiments, the one or more electroactive polymer actuators may be arranged circumferentially around the expandable structure or form a band surrounding a circumference of the expandable structure. In embodiments, one or more electroactive polymer actuators are located adjacent the expandable structure, for example on the inner surface of the shaft of the occlusion device, on the outer surface of the shaft of the occlusion device, or embedded in the shaft of the occlusion device, on the inner surface of the catheter wall, on the outer surface of the catheter wall, or embedded in the catheter wall.

In embodiments, the distal end portion of the device comprises a sleeve positioned over one or more EAP actuators. In embodiments, the one or more EAP actuators are located on the inner surface of the catheter wall, on the outer surface of the catheter wall, or embedded in the catheter wall. In embodiments, the sleeve comprises a cavity. One or more EAP actuators may be located in the cavity, and/or on the inner surface of the sleeve wall, on the outer surface of the sleeve wall, or embedded in the sleeve wall.

In embodiments, the sleeve is an expandable sleeve. In embodiments, the expandable sleeve is an inflatable sleeve. In embodiments, the catheter device comprises at least one inflation lumen in fluid communication with a cavity of the inflatable sleeve. In embodiments, the at least one inflation lumen extends from a proximal end, or proximal end portion of the catheter device, to the inflatable sleeve. In embodiments, the at least one inflation lumen is arranged on the outer surface of the catheter wall, embedded in the catheter wall, or on the inner surface of the catheter wall. In embodiments, the lumens (e.g., the infusion lumen, the aspiration lumen, the inflation lumen for the occlusion device, the inflation lumen for the inflatable sleeve, and any other device lumen mentioned in the present disclosure) are concentrically arranged. In embodiments, the lumens are arranged side-by-side and/or around the longitudinal axis of the catheter body.

In embodiments, the expandable sleeve comprises one or more electroactive polymer actuators configured to expand and/or contract the expandable structure in response to electrical signals. In embodiments, one or more electroactive polymer actuators are located on an inner surface of the expandable structure, on an outer surface of the expandable sleeve, and/or are embedded in the expandable sleeve. In embodiments, the one or more electroactive polymer actuators may be arranged circumferentially around the expandable sleeve or form a band surrounding a circumference of the expandable sleeve. In embodiments, one or more electroactive polymer actuators are located adjacent the expandable sleeve, for example on the inner surface of the catheter wall, on the outer surface of the catheter wall, or embedded in the catheter wall.

In embodiments, the sleeve comprises or consists of a thermoplastic material such as silicon, polyurethane, e.g., Tecoflex™. In embodiments, the sleeve is made of a monolayer or a multilayer film.

In embodiments, the distal end portion of the catheter device comprises a composition comprising the one or more therapeutic agents. In embodiments, the catheter device may comprise one or more coatings comprising a composition comprising the one or more therapeutic agents. The coating(s) may be located on the catheter body, on the sleeve and/or one any other components of the catheter device or system. In embodiments, the coating(s) may be located adjacent to or on one or more EAP actuators.

The composition may include a slow and/or controlled release formulation. In embodiments, the composition is formulated to release the one or more therapeutic agents in response to electrical signals, EAP mechanical or vibrational motion, heat and/or fluid agitation.

In embodiments, the catheter device is configured to release heat from the distal portion. In embodiments, the distal portion of the catheter device is configured to generate fluid agitation and or fluid turbulence. In embodiments, the catheter device comprises one or more gas outlet ports and one or more gas lumen coupled to said gas outlet ports. In embodiments, a system is provided which comprises a catheter device according to the present disclosure and a separate heating and/or agitation device.

According to a further aspect of the invention, there is provided a catheter device comprising: a proximal end portion coupled to a controller configured to provide electrical signals; and a distal end portion comprising one or more electroactive polymer actuators configured for vibrational motion in response to the electrical signals; wherein the catheter device is configured to carry the electrical signals from the controller to the one or more electroactive polymer actuators; and wherein the distal end portion of the catheter device further comprises a sleeve positioned over the one or more EAP actuators.

In embodiments, the sleeve is mechanically decoupled from the one or more actuators.

In embodiments, the sleeve is an expandable sleeve.

In embodiments, the one or more EAP actuators are located on an inner surface of the catheter wall, on an outer surface of the catheter wall, and/or are embedded in the catheter wall. In embodiments, two or more EAP actuators may be arranged radially along a circumference of the catheter body, longitudinally along the catheter body, or helically around the catheter body.

In embodiments, the one or more EAP actuators are located on an inner surface of the sleeve, on an outer surface of the sleeve, or and/or are embedded in a sleeve wall. In embodiments, two or more EAP actuators may be arranged radially along a circumference of the sleeve, longitudinally along the sleeve, or helically around the sleeve.

In embodiments, the expandable sleeve is an inflatable sleeve. In embodiments, the catheter device comprises at least one inflation lumen in fluid communication with a cavity of the inflatable sleeve. In embodiments, the at least one inflation lumen extends from a proximal end, or proximal end portion of the catheter device, to the inflatable sleeve. In embodiments, the at least one inflation lumen is arranged on the outer surface of the catheter wall, embedded in the catheter wall, or on the inner surface of the catheter wall. In embodiments, the lumens (e.g., the infusion lumen, the aspiration lumen, the inflation lumen for the occlusion device, the inflation lumen for the inflatable sleeve, and any other device lumen mentioned in the present disclosure) are concentrically arranged. In embodiments, the lumens are arranged side-by-side and/or around the longitudinal axis of the catheter body.

In embodiments, the catheter device comprises one or more EAP actuators configured to transition the expandable sleeve between a first collapsed configuration and a second expanded configuration, in response to electrical signals provided by the/a controller. In embodiments, one or more electroactive polymer actuators are located on an inner surface of the expandable structure, on an outer surface of the expandable sleeve, and/or are embedded in the expandable sleeve. In embodiments, the one or more electroactive polymer actuators may be arranged circumferentially around the expandable sleeve or form a band surrounding a circumference of the expandable sleeve. In embodiments, one or more electroactive polymer actuators are located adjacent the expandable sleeve, for example on the inner surface of the catheter wall, on the outer surface of the catheter wall, or embedded in the catheter wall. Individual controllers for each component, or one controller to control the components selectively.

In embodiments, the sleeve comprises or consists of a thermoplastic material such as silicon, polyurethane, e.g., Tecoflex™. In embodiments, the sleeve is made of a monolayer or a multilayer film.

In embodiments, the catheter device is further configured to release one or more therapeutic agents. In embodiments, the sleeve comprises a composition comprising the one or more therapeutic agents. In embodiments, the sleeve may comprise one or more coatings comprising a composition comprising the one or more therapeutic agents.

The composition may include a slow and/or controlled release formulation. In embodiments, the composition is formulated to release the one or more therapeutic agents in response to electrical signals, EAP mechanical or vibrational motion, heat and/or fluid agitation.

Features of catheter device according to the first aspect may be combined with features of the catheter device according to the second aspect. Features of catheter device according to the second aspect may be combined with features of the catheter device according to the first aspect.

According to another aspect of the present disclosure, there is provided a method for the delivery of a medical device into a patient, comprising the step of introducing the medical device in a lumen of a catheter device comprising: a proximal end portion coupled to a controller configured to provide electrical signals; and a distal end portion comprising two or more electroactive polymer actuators configured for mechanical or vibrational motion in response to the electrical signals; wherein the electrical signals are provided in a predetermined coordinated pattern.

In embodiments, the predetermined coordinated pattern comprises two or more groups of the electroactive polymer actuators being activated sequentially.

In embodiments, the predetermined coordinated pattern comprises two or more groups of the electroactive polymer actuators being activated concurrently.

In embodiments, two or more electroactive polymer actuators extend in a longitudinal direction relative to the catheter device.

In embodiments, two or more electroactive polymer actuators extend in a radial direction relative to the catheter device.

In embodiments, the method includes the step of pushing the medical device along a lumen of the catheter device through a peristaltic motion.

In embodiments, the method includes the step of pushing the medical device along a lumen of the catheter device through a stick-slip motion.

According to another aspect of the present disclosure, there is provided a method for delivering energy to a target area in a patient comprising the step of using a catheter device comprising: a proximal end portion coupled to a controller configured to provide electrical signals; and a distal end portion comprising one or more electroactive polymer actuators configured for vibrational motion in response to the electrical signals; wherein the catheter device is configured to carry the electrical signals from the controller to the one or more electroactive polymer actuators. In embodiments, the catheter device is a catheter device according to the present disclosure.

According to another aspect of the present disclosure, there is provided an atherectomy method for the disruption of plaque from a patient's vasculature, comprising the step of using a catheter device comprising: a proximal end portion coupled to a controller configured to provide electrical signals; and a distal end portion comprising one or more electroactive polymer actuators configured for vibrational motion in response to the electrical signals; wherein the catheter device is configured to carry the electrical signals from the controller to the one or more electroactive polymer actuators. In embodiments, the catheter device is a catheter device according to the present disclosure.

According to another aspect of the present disclosure, there is provided a method for the delivery of one or more therapeutic agents to a patient, comprising the step of using a catheter device according to the present disclosure.

According to another aspect of the present disclosure, there is provided a method for preventing the formation of biofilm on a medical device, comprising the step of using a catheter device comprising: a proximal end portion coupled to a controller configured to provide electrical signals; and a distal end portion comprising one or more electroactive polymer actuators configured for vibrational motion in response to the electrical signals; wherein the catheter device is configured to carry the electrical signals from the controller to the one or more electroactive polymer actuators. In embodiments, the catheter device is a catheter device according to the present disclosure.

According to another aspect of the present disclosure, there is provided a method for modifying a patient's tissue or lumen comprising the step of using a catheter device comprising: a proximal end portion coupled to a controller configured to provide electrical signals; and a distal end portion comprising one or more electroactive polymer actuators configured for vibrational motion in response to the electrical signals; wherein the catheter device is configured to carry the electrical signals from the controller to the one or more electroactive polymer actuators. In embodiments, the catheter device is a catheter device according to the present disclosure.

In embodiments, the modification includes one or more of:
increasing nitric oxide production;
increasing or decreasing vasodilation;
increasing adenosine triphosphate (ATP) production;
reducing inflammatory response; and
increasing or decreasing angiogenesis.

According to another aspect of the present disclosure, there is provided a method for detecting a characteristic or configuration of a patient's lumen comprising the step of using a catheter device comprising: a proximal end portion coupled to a controller configured to provide electrical signals; and a distal end portion comprising one or more electroactive polymer actuators configured for vibrational motion in response to the electrical signals; one or more sensors; wherein the catheter device is configured to carry the electrical signals from the controller to the one or more electroactive polymer actuators. In embodiments, the catheter device is a catheter device according to the present disclosure.

In embodiment, the characteristic or configuration includes one or more of:
Stenosed area(s) of the lumen;
Presence of a thrombus;
Wall contact between one or more actuator(s) and the lumen; and
Pressure changes.

According to another aspect of the present disclosure, there is provided a method for the stimulation of a patient's nervous system comprising the step of using a catheter device comprising: a proximal end portion coupled to a controller configured to provide electrical signals; and a distal end portion comprising one or more electroactive polymer actuators configured for vibrational motion in response to the electrical signals; wherein the catheter device is configured to carry the electrical signals from the controller to the one or more electroactive polymer actuators. In embodiments, the catheter device is a catheter device according to the present disclosure.

According to another aspect of the present disclosure, there is provided a method for the delivery of a medical device into a patient, comprising the step of using, as a delivery device, a catheter device comprising: a proximal end portion coupled to a controller configured to provide electrical signals; and a distal end portion comprising one or more electroactive polymer actuators configured for vibrational motion in response to the electrical signals; wherein the catheter device is configured to carry the electrical signals from the controller to the one or more electroactive polymer actuators. In embodiments, the catheter device is a catheter device according to the present disclosure.

In embodiments, the method comprises the step of reducing friction between the catheter device and the medical device.

Other aspects, features and advantages of the present application will be appreciated from the following detailed description, the accompanying drawings, and the appended claims.

### DESCRIPTION OF DRAWINGS

The details of one or more implementations are set forth, by way of example only, in the accompanying drawings and the description below. Other features, aspects, and advantages of the disclosure will become apparent from the description, the drawings, and the claims, in which:

FIG. 1 is a schematic representation of the distal end portion of a catheter device according to the present invention;

FIG. 2 is a schematic cross sectional representation of the distal end portion of a catheter device according to the present invention;

FIG. 3 is a schematic representation of the distal end portion of a catheter device according to the present invention;

FIGS. 4A to 4C each illustrate one of three activation patterns (sequentially, simultaneously, or grouped, respectively), under each activation pattern, four activation control signals (201a-201d) correspond to the control signals sent to actuators 201a-201d;

FIG. 5 is a schematic representation of the distal end portion of a catheter device according to the present invention;

FIG. 6 is a schematic representation of the distal end portion of a catheter device according to the present invention, for the delivery of one or more therapeutic agents;

FIG. 7 is schematic representation of the distal end portion of a catheter device according to the present invention, comprising an occlusion device;

FIG. 8 is schematic representation of the distal end portion of a catheter device according to the present invention, comprising a sleeve;

FIG. 9 is schematic representation of the distal end portion of another catheter device according to the present invention, comprising a sleeve;

FIG. 10 is schematic representation of the distal end portion of another catheter device according to the present invention, comprising a sleeve;

FIG. 11 is schematic representation of the distal end portion of another catheter device according to the present invention, comprising a sleeve;

FIG. 12 is schematic representation of the distal end portion of another catheter device according to the present invention, comprising a sleeve; and

FIG. 13 is schematic representation of the distal end portion of another catheter device according to the present invention, comprising a sleeve and an occlusion device;

The embodiments described herein are provided as exemplary and non-limiting embodiments of the present invention.

### DETAILED DESCRIPTION

The present disclosure relates to various embodiments of a medical or surgical device comprising electroactive polymer actuators for delivering energy to a target site in a patient. As will be illustrated in more detail hereinbelow, the target site may be part of the patient's anatomy (e.g., anatomical tissue, organ, vasculature, vessel, or one or more specific portion thereof) or may be part of another device; the energy may be delivered to a specific target site so as to impact a substance or composition in situ.

The catheter devices according to the present disclosure may include a distal end portion, which may be actuated to vibrate vigorously to achieve desirable effects suitable for use in various surgical applications (e.g., thrombectomy, atherectomy, drug delivery, device delivery, biofilm prevention, neurostimulation, and other applications as illustrated below). Unlike devices in minimally invasive surgery, where the tissues subject to the procedure are accessed through conveniently located small incisions, a location in a blood vessel is typically accessed through a long flexible catheter, often 100 cm or more in length. However, while this detailed description provides examples of implementing the present invention in conjunction with a catheter, the present invention may be implemented even in minimally invasive surgical devices as well.

One or more electroactive polymer (EAP) actuators may be provided at the distal end portion (also referred to as “distal tip”) of the catheter device. Each EAP actuator may be actuated (e.g., set in motion or vibration) by electrical signals transmitted from the proximal end or proximal end portion of the instrument. In this arrangement, the mechanical or vibrational motion of the distal tip may be confined and not transferred to any substantial length of the instrument.

In some embodiments, the actions of the EAP actuators need not be linear. The linear motion may be supplemented by other mechanical motion of the distal tip. For example, in some embodiments, the range of mechanical motions at the distal tip may include any radial, axial, torsional and helical motion, or combination thereof. In this context, a torsional motion refers to a rotational motion about the axial direction and a helical motion refers to a combination of torsional and axial motions.

Suitable electroactive polymers include various combinations of vinylidene fluoride (VDF), trifluoroethylene (TrFE), 1,1-chlorofluoroethylene (CFE), and chlorotrifluoroethylene (CTFE). For example, the terpolymers P(VDF-TrFE-CTFE) and P(VDF-TrFE-CFE) are available commercially from Piezotech (a subsidiary of Arkema S.A., Paris, France). These terpolymers, which have different electroactive properties, exhibit large electrostrictive strain (e.g., greater than 0.5%, preferably greater than 3.0%) under electric fields of 20-200 V/um (e.g., 20-100V/μm; preferably, about 50V/um).

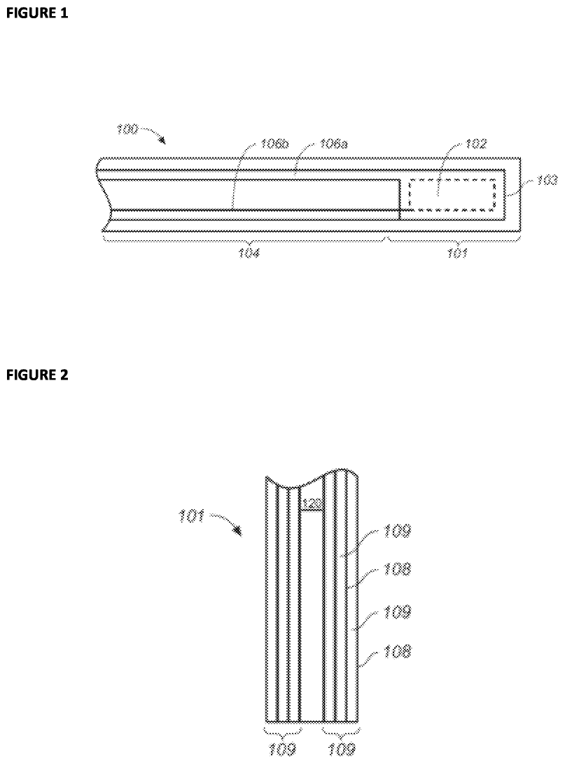

FIG. 1 is a schematic view of a catheter device 100 having a distal tip 101 and catheter body or shaft 104. The distal tip 101 may be itself an actuator or includes one or more actuators that are each capable of electrically controlled motion. The device 100 includes a proximal end with a watertight connection to an electronic signal generator and a controller.

The catheter body 104 may be of a conventional mechanical design, such as having an inner layer or liner of poly-tetrafluroethylene (PTFE), Pebax or thermoplastic polyurethane (TPU). The PTFE inner layer may be surrounded by an outer layer of a reflowable material (e.g., Pebax with varying durometers across the length of the catheter body 104). In addition, the catheter body 104 accommodates both an active electrode 106a and a return electrode 106b, which are electrically insulated from each other, each electrode extending along the entire length of the catheter body 104. These electrodes may be formed out of any suitable electrically conductive wires. The inner layer or the conductive wires may be provided with suitable mechanical strength, or in the form of a braid or coil, so as to provide the catheter body 104 mechanical integrity and kink resistance. The conductive wires may be embedded in an electrically non-conductive braid or a coil (e.g., constructed from poly-ether-ether ketone (PEEK)) that extends along the entire length of device 100. These braids or coils are available in various patterns from, for example, Steeger USA, US Biodesign, Inc., and Admedes, Inc. Alternatively, an all-metallic braid or coil with electrically insulated wires for the active electrode 106a and the return electrode 106b are also possible. However, embedding the electrodes in a non-conductive braid or coil is preferable to avoid shorting. Although, purely for illustrative purpose, only the active electrode 106a and the return electrode 106b are shown in FIG. 1; any suitable number of active electrodes and return electrodes may be used.

The lumen 120 of the catheter device 100 runs substantially the entire length of the catheter body 104. The lumen 120 may be a multi-lumen conduit configured to accommodate a plurality of lumen, including but not limited to drug infusion lumen, inflation lumen for inflatable components, aspiration lumen for the intake of occlusion fragments, and the like, depending on the intended application.

Layers of the EAP are embedded inside the distal tip 101. Each EAP layer strains when an electric field is placed across it. (Note that, although a greater strain is achieved at a greater electric field, the strain-electric field relationship is generally non-linear.) As shown in FIG. 1, the EAP layers are each provided between thin and flexible layers of electrodes, e.g., between electrode 102 and electrode 103, which is underneath electrode 102. Electrodes 102 and 103 are each electrically connected to either active electrode 106a or return electrode 106b. In this manner, movement occurs only at distal tip 101 at the distal end of the device 100 and no energy is lost in moving the active electrode 106a and the return electrode 106b in catheter body 104. In one embodiment, each EAP layer may be between 2-20 um thick. The distal tip 100 may move in both the longitudinal direction and in the transverse direction.

According to one embodiment of the present invention, each EAP layer may be formed by dip-coating. For example, the distal tip 101 may be dipped in a solution of the EAP in a polar solvent, such as diethyl formamide (DMF) or methyl ethyl ketone (MEK). In this manner, coaxial 20-200 μm thick EAP layers may be formed in the distal tip 101 in successive dips. After forming each EAP layer, an electrode layer is formed over the exposed surface of the EAP layer by, for example, sputtering (e.g., gold or aluminum), clip-coating (e.g., silver-embedded urethane), pad printing or spray coating using a conductive electric ink or a particle-free metal-complex conductive ink (e.g., conductive inks available from Electroninks or LiquidX). The forming steps for the EAP layer-electrode layer combination may be repeated multiple times. The electrode layers thus formed may be connected to either active electrode 106a or return electrode 106b, such that electrodes of opposite polarities are formed on opposite sides of an EAP layer, creating in effect a capacitor.

FIG. 2 is a cross-section, transverse to the cross-section of FIG. 1, of the distal tip 101, showing electrode layers 108 and EAP layers 109. Depending on the mechanical properties desired, each EAP layer may have any one of various thicknesses. Additional non-EAP layers (not shown) may also be included. The electrodes to the EAP layer or layers of the actuators may be individually provided. Multiple actuators may be integrated into the tip of the instrument as straight sections and actuated independently.

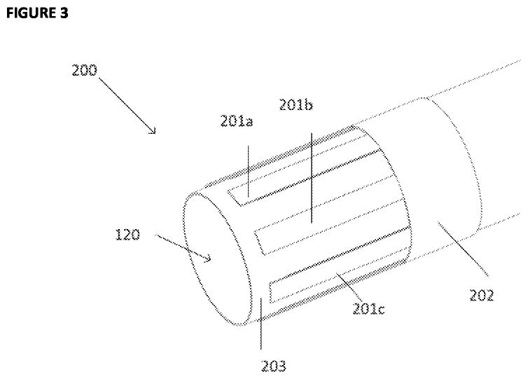

FIG. 3 illustrates a distal tip 200 that includes multiple individually controlled actuators (e.g., actuators 201a-201d; actuator 201d situated on opposite side and thus not seen in FIG. 3). In this configuration, each actuator may be an axially or longitudinally aligned beam encapsulated at its proximal end to circumferentially wrapped substrate layer 202. As shown in FIG. 3, inner layer 203 of a compliant polymer material may be provided to line the lumen 120. Circumferentially wrapped substrate layer 202 overlays both the proximal ends of the actuators and inner compliant layer 203. The substrate layer 202 may be formed of a high modulus material relative to the material in the immediate surrounding (e.g., compliant layer 203). Nonetheless, the layer 203 may have a higher modulus of elasticity relative to conventional backing materials in order to improve characteristics such as bendability, flexibility, expansion or contraction of the diameter of the catheter body.

EAP actuators may be integrated with other mechanical or electrical elements into an actuator (“integrated EAP actuator”) that can be used as a building block for constructing an instrument. An integrated EAP actuator may have characterized electromechanical properties and may be formed to have any desired geometry for deployment in a medical device (e.g., distal end portion of catheter device 100). The EAP actuators may be formed on an outer surface of the catheter wall or on an inner surface of the catheter wall, or may be partially or completely embedded in a catheter wall. The EAP actuators may be configured as circumferential rings, circumferentially arranged strips and/or axial strips. Thus, one or more integrated EAP actuators may be incorporated into distal tip 100 (e.g., as a three-dimensional array of integrated actuators).

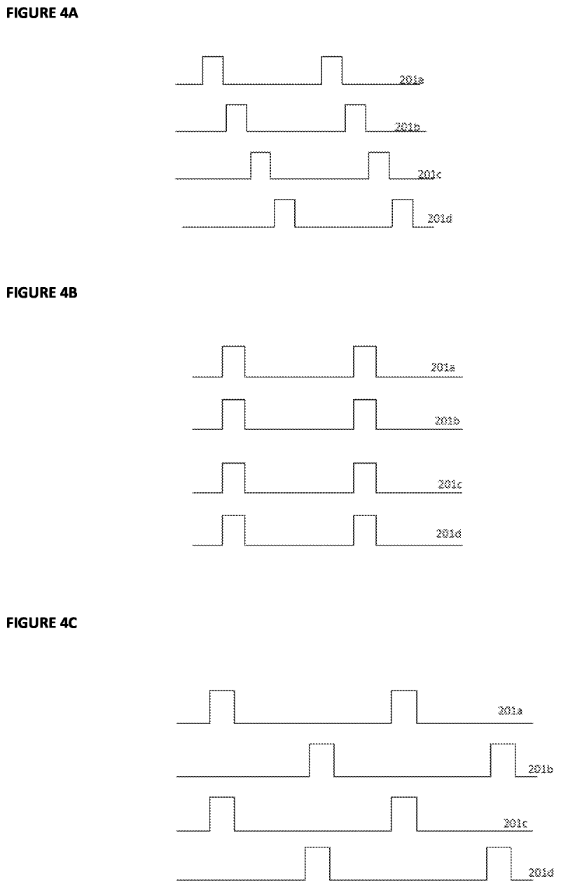

The EAP actuators may be in electrical communication with and operated by one or more controllers, electrical signal generators, and/or drivers, integrated or external. The EAP actuators can operate, vibrate and/or move at subsonic frequencies (0-20 Hz), sonic frequencies (20-20,000 Hz) or ultrasonic frequencies (20 Hz or greater). Using a combination of actuation patterns, a medical professional can drive the EAP actuators individually and collaboratively, to achieve various effects. FIGS. 4A to 4C illustrate the control signals to actuators 201a-201c under a sequential activation pattern, a concurrent or unison actuation pattern, and a coordinated or grouped actuation pattern, respectively. In FIG. 4A, for example, under the sequential actuation pattern, actuators 201a-201b are each actuated according to a predetermined order. In FIG. 4B, under a concurrent or unison actuation pattern, actuators 201a-201b are actuated simultaneously. In FIG. 4C, under a coordinated actuation pattern, actuators 201a and 201c and actuators 201b and 201d form two groups that are actuated sequentially. Within 15 each group, however, the actuators are actuated simultaneously. In embodiments having even more actuators, an even more complex but suitable actuation pattern can be constructed. Of importance, the actuators can be activated by combining actuation patterns into a sequence of actuation patterns to tailor a desirable specific result.

Each of the embodiments described herein may be driven by a drive electronic circuit. If the distal tip 101 is designed to have multiple independently controlled actuators, more than one waveform may be provided to each of the active electrodes. The drive circuit may provide driving waveforms, for example, between 50.0-250.0 volts (peak-to-peak). The driving waveform may be sinusoidal, triangular, square or any desired wave shape (preferably, a square wave, such as shown in FIG. 4A to 4C) to provide the greatest acceleration or vibration. A suitable driving circuit may be provided, for example, using Microchip HV56020 or Microchip HV 56022.

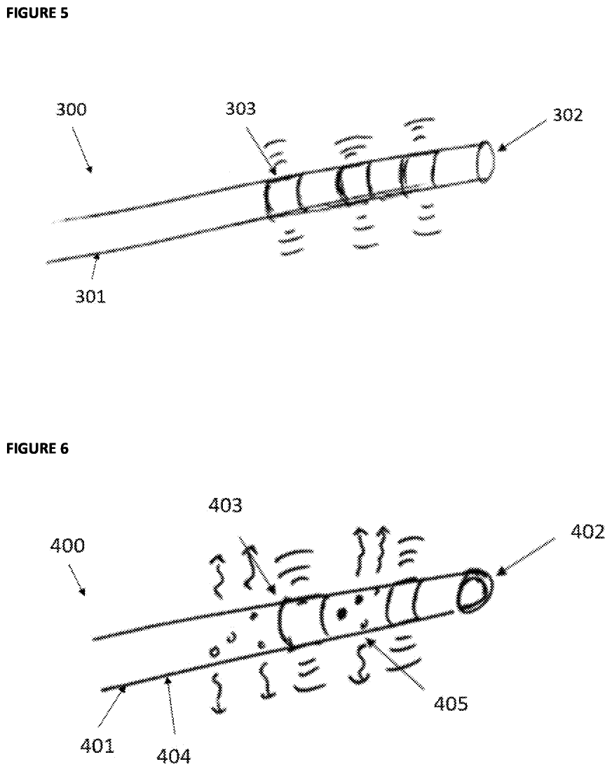

FIG. 5 illustrates another catheter device 300 comprising a catheter body 301 and a distal opening 302 at the distal end of the catheter body 301. Devices according to the present disclosure may comprise two or more EAP actuators. In this embodiment, the catheter device 300 comprises three EAP actuators 304 in the shape of circumferential rings and spaced along the distal end portion of the catheter device 300. As noted hereinabove, the EAP actuators may have various configurations.

The EAP actuators 304 may be configured to vibrate at one or more frequencies to transfer energy from the EAP actuators 304 to surrounding tissue, to transfer energy from surrounding tissue to the EAP actuators, and/or to modulate the shape of at least one portion of the catheter body 301 (for example, of the distal portion of the catheter device 300).

The catheter device 300 and other catheter devices as described in the present application may be used to prevent the formation of biofilm and/or growth of bacteria which has a tendency to accumulate on indwelling devices, in particular on long-term intracorporeal devices such as long-term catheters, percutaneous endoscopic gastrostomy (“PEG”) tubes, peripherally inserted central catheter (PICC) lines. In response to electrical signals at frequencies from low sonic to ultrasonic, the EAP actuators generate a vibrational motion of the catheter device which may reduce or prevent the adherence of any material (such as biofilm, bacteria, salts and the like) on the surface of the device, or eliminate and remove said materials from the surface of the device.

The catheter device 300 and other catheter devices as described in the present application may be used in atherectomy treatments, or any procedures requiring the breakdown of calcification in the vasculature (e.g., the pathological deposition of minerals in the vascular system) at any time during development. In response to electrical signals at sonic frequencies, the EAP actuators generate a vibrational motion of the catheter device which may disrupt plaques accumulating in the patient's vessels (for example, in the arteries).

The catheter device 300 and other catheter devices as described in the present application may be used to stimulates a patient's nervous system or parts thereof. The EAP actuators may provide mechanical or vibratory stimulation to one or more nerves adjacent to or contacting the physiological lumen (or vessel) in which the catheter device is positioned. The stimulation may be acoustic, ultrasonic, or the like.

The catheter device 300 and other catheter devices as described in the present application may be used to modify a characteristic and/or property of a part of the patient's anatomy, for example patient's tissue or physiological lumen (e.g., an artery, vein, duct, ureters, and the like). Energy may be transmitted to a target site at one or more frequencies for example to increase nitric oxide production, impact vasodilation (e.g., increase or reduce vasodilation depending on the one or more frequencies), increase adenosine triphosphate (ATP) production, reduce inflammatory response(s), and/or increase or reduce angiogenesis.

The catheter device 300 and other catheter devices as described in the present application may be used as a delivery device. This may be achieved by modifying the friction characteristics (for example, by reducing the friction) between the catheter wall and the device to be delivered. In embodiments, the EAP actuators are configured to radially (or contract) expand the catheter lumen, thereby decreasing (or increase) the friction between the catheter wall and the device to be delivered. In embodiments, two or more discrete segments of the catheter wall may radially expand and/or contract in a predetermined pattern such that the intracorporeal device is pushed along the catheter lumen through a peristaltic motion. In embodiments, two or more discrete segments of the catheter wall may axially expand and/or contract in a predetermined pattern such that a first segment of the catheter wall moves relative to the intracorporeal device while a second segment remains stationary. In embodiments, friction between the catheter wall and the intracorporeal device may be reduced by creating a stick-slip motion through high frequency axial vibrations.

The catheter device may form part of a delivery system for the delivery of a wide variety of medical devices, including intracorporeal devices such as stents, coils, implants, valve replacement devices, valve repair devices, septal repair devices, and the like, and other delivery tools, such as catheters, guidewires, dilators, guiding tools, imaging tools and the like. According to the present disclosure, there is provided a delivery system comprising at least one catheter device as described in the present application. The system may comprise an intracorporeal device and/or a delivery tool.

The catheter devices as described in the present application may comprise one or more sensors, such as temperature sensors, pressure sensors flow rate sensors, and the like. Alternatively or additionally, the catheter device may comprise a plurality of EAP actuators along the length of the catheter body and configured to signal changes the controller. Such changes may include the narrowing or widening of the physiological lumen (for example, a stenosed area of the physiological lumen would be narrower). The EAP actuators may be configured to detect wall contact (between the physiological wall and the EAP actuators on the outer surface of the catheter device) and/or friction. The one or more EAP actuators may be used as feedback or use with surgical robotics or for physician information. The one or more EAP actuators can be further configured to detect pressure changes. The one or more EAP actuators can be further configured to detect presence of thrombus at tip or inside the lumen of the catheter.

The catheter devices as described in the present application may comprise one or more markers. The catheter device may be comprised in a system that further comprises internal and/or external imaging and/or visualising components. Alternatively or additionally, the EAP actuators may be configured to selectively increase or decrease a segment of the catheter body to assist in tracking said segment. In embodiments, the EAP actuators may be configured to modify the shape of a segment, such as that of the distal end, to track the position of the distal end. For example, the distal end of the catheter device may be modified into a funnel-like shape. The EAP actuators may be used to detect a variety of characteristics, such as the location of one or more segments or portions of the catheter device in the patient, or the detection of substances in the body (e.g., plaque, stenoses, cancerous cells, and the like). The EAP actuators may be configured to improve navigation through the patient's vasculature by modifying the shape, dimensions and/or configuration of the catheter device.

FIG. 6 illustrates a catheter device 400 for the delivery of one or more therapeutic agents comprising a proximal end portion coupled to a controller configured to provide electrical signals; and a distal end portion comprising one or more electroactive polymer actuators 403 configured for vibrational motion in response to the electrical signals; wherein the catheter device 400 is configured to carry the electrical signals from the controller to the one or more electroactive polymer actuators 403; and wherein the catheter device 400 is further configured to release one or more therapeutic agents from the distal end portion of the catheter device 400.

Within the context of the present invention, “proximal end” and “proximal end portion” in respect of a device or component refers to the end and end portion of the device or component closer, in use, to the medical practitioner. The “distal end” and “distal end portion” in respect of a device or component refers to the end and end portion of the device or component closest, in use, to the target site in the patient.

The catheter device 400 comprises a catheter body 401 with a distal end 402, and a plurality of EAP actuators 403 which, in this example, are configured as circumferential bands around catheter wall 404. The catheter device 400 comprises a plurality of infusion ports 405 in fluid communication with one or more infusion lumen.

The infusion ports 405 may be formed radially or longitudinally relative to the catheter body 401, or helically around the catheter body 401. The infusion ports may be grouped for focused infusion or dispersed across a wider surface to treat a broader area.

The infusion lumen extend from the infusion ports 405 to the proximal end or proximal end portion of the catheter device 400. The infusion lumen may be in fluid communication with a source of therapeutic agents. The source is for example a syringe, a bottle, a cartridge, or a bag, and may be integral with the catheter device 400 or provided as a separate component of a delivery system. The catheter device 400 may comprise a plurality of infusion lumen configured to deliver the same therapeutic agent or different therapeutic agents. It is therefore possible to deliver two therapeutic agents which when alone are inactive but which activate upon release into the patient's vessel and contact with each other.

In embodiments, one or more therapeutic agents may be released from the catheter device 400, for example from a coating on a surface of the catheter body 401. The formulation of the coating may be adjusted for slow-release or controlled-release of the one or more therapeutic agents.

In embodiments, one or more therapeutic agents may be in the form of microbubbles. Within the context of the present disclosure, a microbubble comprises a fluid core or a gas core encapsulated in a shell. In embodiments, the microbubbles have a diameter of from 1-10 μm. In embodiments, the microbubbles are configured to release their core in response to vibrational motion of the one or more EAP actuators.

The microbubbles may comprise a core including one or more therapeutic agents and a shell encapsulating said core. The core may, alternatively or additionally comprises a liquid or gas. The shell may comprise or consist of surfactants, proteins, lipids and/or polymers, including denatured proteins, biocompatible polymers, phospholipids, and a combination thereof.

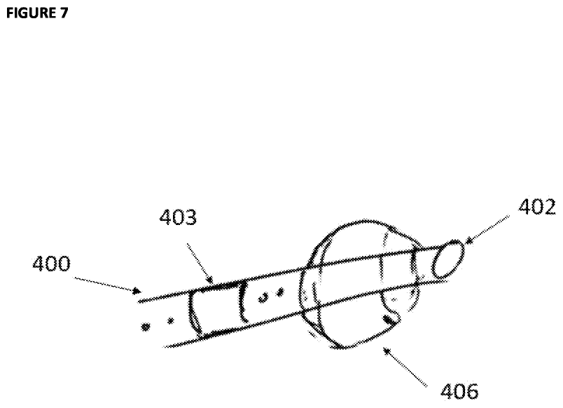

A drug delivery method according to the present disclosure will now be described using the example of an atherectomy treatment. In use, the catheter device 400 is inserted into and pushed through the patient's vasculature, to the plaque. The distal end portion is positioned such that the infusion ports 405 are adjacent or facing the plaque. In embodiments, an occlusion device 406 is deployed at a distal position relative to the target site, as illustrated in FIG. 7. A second occlusion device (not shown) may be deployed at a proximal position relative to the target site, thereby creating an infusion compartment between the first and the second occlusion devices which may be immersed and treated with the therapeutic agent(s).

The therapeutic agent(s) (for example a lytic agent) and/or microbubbles (for example microbubbles with a lytic agent-containing core) may be released through the infusion ports 405 into the patient's lumen and come into contact with the plaque. The therapeutic agent(s) may be active or become active. The therapeutic agent(s) may for example become active or may be released upon application of a trigger. The trigger may be the vibrational motion of the EAP actuators 403 in response to electrical signals provided an electrical signal generator and/or controller. The trigger may be, additionally or alternatively, heat; the catheter device 400 may be configured to heat the therapeutic agent(s) before, upon or after release or to heat the infusion site. The trigger may be, additionally or alternatively, fluid agitation; the catheter device 400 may be configured to generate agitation or turbulence at the infusion site for example by infusion of a gas. The trigger may be, additionally or alternatively, the contacting of two or more therapeutic agent(s) upon release from the infusion ports 405. The trigger may activate or release the therapeutic agent(s) for example from microbubbles. It has also been observed that the described triggers each improve and enhance the therapeutic activity, delivery and distribution of the therapeutic agent(s).

Upon degradation, fragmentation and/or disruption of the plaque, the occlusion device 406 may be folded to allow aspiration of fragments through the distal opening 402 of the catheter device 400. Additionally or alternatively, the catheter device 400 may comprise aspiration ports in the catheter wall 401 and coupled to one or more aspiration lumens so that fragments in the infusion compartment between two occlusion devices may be removed.

Other treatments may be performed using the catheter device 400. In the case of a thrombectomy treatment, the distal end of the catheter device 400 may be configured to pierce and travel through the occlusion. Occlusion devices may be expanded on the distal and proximal sides of the occlusion to isolate the occlusion in an infusion compartment.

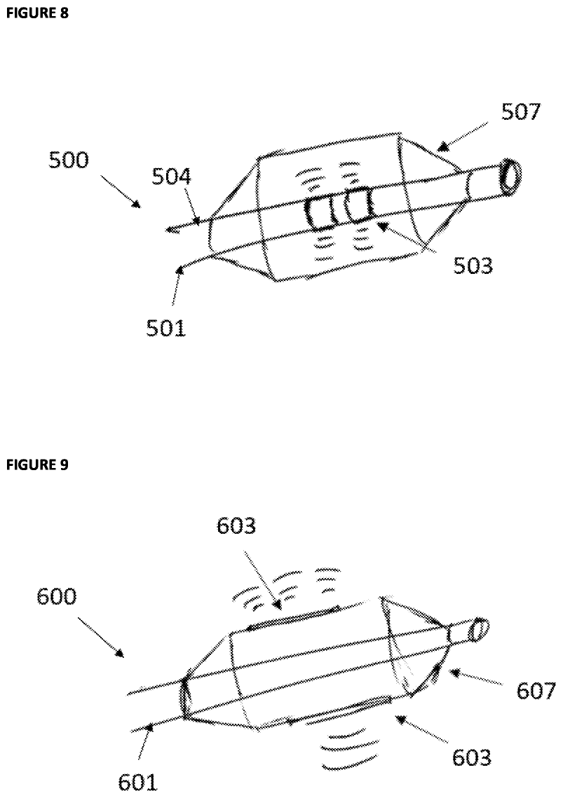

FIG. 8 illustrates a catheter device 500 comprising a proximal end portion coupled to a controller configured to provide electrical signals; and a distal end portion comprising one or more electroactive polymer actuators 503 configured for vibrational motion in response to the electrical signals; wherein the catheter device 500 is configured to carry the electrical signals from the controller to the one or more electroactive polymer actuators 503; and wherein the distal end portion of the catheter device 500 further comprises a sleeve, such as an expandable sleeve 507.

The EAP actuators 503 may be located on an inner surface of the catheter wall 504, on an outer surface of the catheter wall 504, and/or are embedded in the catheter wall 504. In this embodiment, the EAP actuators 503 are located on outer surface of the catheter wall 504. Upon receiving electrical stimulation, the EAP actuators 503 vibrate.

The vibrational motion, and hence energy, may be redirected from the EAP actuators 503 in a preferential direction. The shape and configuration of the sleeve 507 may be set, adjusted and/or controlled to redirect the vibrational motion in the intended direction. The vibrational motion may be transmitted to the surface of the expanded sleeve 507, and the energy transmitted to the patient's lumen wall (or tissue), to disrupt plaque or to modify one or more characteristics of the patient's lumen wall (or tissue), as described hereinabove.

The vibrational motion may be transmitted to the surface of the expanded sleeve 507 which may be coated with a composition comprising one or more therapeutic agents. The vibrational motion may activate or energise the therapeutic agent(s) and/or release the therapeutic agent(s) from the composition. The vibrational motion may increase the uptake of the therapeutic agent(s) by the patient's lumen wall (or tissue) in contact with the expanded sleeve 507.

FIG. 9 illustrates another catheter device 600 comprising a proximal end portion coupled to a controller configured to provide electrical signals; and a distal end portion comprising one or more electroactive polymer actuators 603 configured for vibrational motion in response to the electrical signals; wherein the catheter device 600 is configured to carry the electrical signals from the controller to the one or more electroactive polymer actuators 603; and wherein the distal end portion of the catheter device 600 further comprises an expandable sleeve 607. The expandable sleeve 607 may be an expandable or inflatable balloon.

The EAP actuators 603 may be located on an inner surface of the sleeve 607, on an outer surface of the sleeve 607, or and/or are embedded in a sleeve wall. In this embodiment, the EAP actuators 603 are located on outer surface of the sleeve 607.

The vibrational motion, and hence energy, may be transmitted to the patient's lumen wall (or tissue), to disrupt plaque or to modify one or more characteristics of the patient's lumen wall (or tissue), as described hereinabove.

The surface of the expanded sleeve 607 may be coated with a composition comprising one or more therapeutic agents. The vibrational motion may activate or energise the therapeutic agent(s) and/or release the therapeutic agent(s) from the composition. The vibrational motion may increase the uptake of the therapeutic agent(s) by the patient's lumen wall (or tissue) in contact with the expanded sleeve 607.

In an alternative embodiment, the sleeve 607 may not be expandable. The sleeve 607 may be positioned over one or more EAP actuators 603. A purpose of the sleeve 607 (expandable or not) located over EAP actuators is to protect the EAP actuators from the surrounding liquid environment in the patient. In embodiments, the sleeve 607 is mechanically decoupled from the EAP actuators 603. Within the context of the present disclosure, “mechanically decoupled” means sleeves which are not bonded or welded onto the EAP actuators; it may include sleeves which are not in contact with the EAP actuators or which are merely resting over the EAP actuators. In embodiments, the sleeve 607 may be formed by applying a film (such as a thermoplastic film) over the catheter body (and the EAP actuators located on the catheter body) and by welding or laminating the film onto the catheter body proximally and distally from the EAP actuators. In other words, the film is not bonded, welded, or laminated onto the EAP actuators.

FIGS. 11 to 13 illustrate additional or alternative features of a catheter device according to the present invention and comprising a sleeve covering the EAP actuators. The figures depict a catheter device 700 comprising a sleeve 707 covering EAP actuators 703 located on the catheter body.

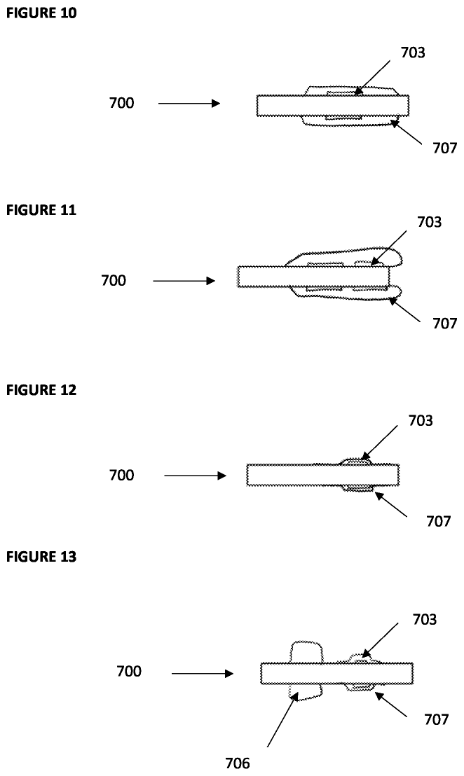

In FIG. 10, the sleeve 707 may be a low-profile sleeve or balloon, such as a low-profile expandable sleeve or balloon. The low profile allows access to narrower vessel. The low profile may also enable flow arrest in said vessel and act as a balloon guide or occlusion balloon, and/or provide catheter stability.

In FIG. 11, the sleeve 707 may be an expandable sleeve (e.g., an expandable or inflatable balloon), which when expanded is dimensioned and shaped to provide an atraumatic distal end. For example, the sleeve 707 may be configured such that the distal end of the expanded sleeve 707 extends distally beyond the distal end of the catheter body.

In FIG. 12, the sleeve 707 may be a non-expandable sleeve 707, which is mechanically decoupled from the EAP actuators 703, as explained hereinabove. In this configuration, the sleeve 707 protects the EAP actuators from the surrounding liquid environment in the patient.

FIG. 13 depicts a catheter device 707 comprising a sleeve 707 and a proximal occlusion device 706.

The catheter devices as described in the present application comprise EAP actuators configured to deliver energy, in the form of a vibrational motion, in response to electrical stimulation. The catheter devices may further comprise EAP actuators configured to modify the shape of one or more components or portions of the catheter device, by mechanical motion in response to electrical stimulation. For example, EAP actuators may be configured to modify the shape, dimension and/or configuration of the catheter body (or parts thereof) to assist in navigating the device through the patient's vasculature. EAP actuators may be configured to open and close the distal opening of a catheter device to allow or prevent aspiration and/or infusion through the central catheter lumen. EAP actuators may be configured to expand and contract an occlusion device or an expandable structure such as a balloon or a sleeve.

The catheter devices as described in the present application are versatile and may be used in applications wherein one or more of the following effects are required: vibration-induced opening of the endothelium, through cavitation and/or acoustic streaming, enhanced vasodilation through endogenous production of nitric oxide, softening and remodeling of the vessel tissue (e.g., collagen), reduction of inflammatory response, and/or targeted delivery of therapeutic agent(s) to an angioplasty site through the lumen (e.g., anaesthetic).

Thus, the present invention provides improves alternatives to existing devices, systems and methods. The devices and systems according to the present disclosure are configured to deliver energy to a target site in a patient and/or on intracorporeal devices. Based on this concept and on the configurations described in the present disclosure, the devices and systems may be used in a wide variety of treatments and procedures.

### Non-Limiting Concepts

Broadly, the present disclosure relates to devices, systems and methods for delivering energy from an actuator incorporated into a medical instrument which may be used anywhere in the body to impact the surrounding environment. The energy may be of any type, such as mechanical, ultrasonic, acoustic and the like. The medical instrument may be for example an endovascular device or a catheter. The concepts according to the present disclosure may be applied to other medical instruments, such as devices that can be inserted and/or implanted into any physiological lumen. The medical instrument is preferably used within a lumen (such as an artery, vein, duct and the like). The impact may include triggering an effect such as vasodilation, visibility, drug delivery, neurological stimulation, polymerization, mechanical disruption of plaque.

The actuators according to the present disclosure may be integrated in a catheter shaft.

A catheter according to the present disclosure may include one or more of the following features:
A flexible tube with a proximal tip, a distal tip, and a shaft extending from the proximal tip to the distal tip, where both distal and proximal tips have at least one opening.
One or more lumens for passing fluid (e.g., lytic agent, therapeutic agent, etc.) or guidewire through at least part of the catheter.
One or more electroactive polymer (EAP) actuators integrated into length of the catheter shaft:
   a. in electrical communication with a controller and/or electrical signal generator
   b. configured to be activated by application of an electric field and/or electric signal to move
   c. each of the one or more EAP Actuators can operate, vibrate, and/or move at Subsonic (020 Hz), sonic (20-20,000 Hz), or ultrasonic frequencies (20 kHz or greater). If more than one, the EAP actuators may operate independently and/or together. The EAP actuators may operate in patterns.
The interior of the flexible tube (e.g., the backing) may have a higher modulus of elasticity compared to traditional backing materials to create/enhance at least one of bending, flexibility, expansion and/or contraction of the diameter of the tube, etc.,

In an embodiment as illustrated in FIG. 5, a catheter tube includes three EAP actuators (but can be any number greater than 1) spaced along the length of the shaft and configured to vibrate at one or more frequencies to (1) transfer energy from the EAP actuators to surrounding tissue (not shown), (2) transfer energy from surrounding tissue to the EAP actuators, and/or (3) modulate the shape of at least a portion of the catheter tube.

### Enhanced Thrombolytics—Ultrasound-Assisted Catheter-Directed Thrombolysis (UACDT)

The catheter according to the present disclosure may be used to inject a lytic agent (e.g. (Alteplase) tPA) into a thrombus (e.g., pulmonary embolism (PE), deep vein thrombosis (DVT), or other) through one or multiple lumens, where the one or more EAP actuators are configured to enhance the lytic effect and may improve transport of the lytic agent.

### Ultrasound Targeted Microbubble (UTMB) Therapeutic Agent Delivery

In an embodiment as illustrated in FIG. 6, a catheter includes at least one infusion port along the length of the catheter shaft near the one or more EAP actuators (two shown, but any number greater than one contemplated), in conjunction with ultrasound targeted microbubbles to provide localized delivery of a therapeutic agent. In embodiments, ultrasound targeted microbubbles may be provided by the catheter, injected intravenously, and/or provided by any other known means (local or systemic). In embodiments, a catheter according to the present disclosure can be used (e.g., opened) to locally activate a therapeutic agent. In embodiments, the therapeutic agent may a drug, a solution, an embolic material (e.g., glue), and/or any other material used for a therapeutic purpose.

Microbubbles may hold the therapeutic agent and may be transported to target area(s) and opened by the application of ultrasound waves (generated by the EAP actuator(s) vibrating at ultrasonic frequencies).

In an embodiment as illustrated in FIG. 7, a catheter includes at least one infusion port and an inflatable balloon on the distal or proximal end (depending on the direction of blood flow relative to catheter introduction) to temporarily occlude blood flow in a vein or artery to allow uptake of the released therapeutic agent before it is washed away from the target location.

### Biofilm Prevention

The catheter according to the present disclosure, or any other configuration of catheter including one or more EAP actuators, may be used by moving the one or more EAP actuators at frequencies from low sonic to ultrasonic, to create vibratory movement of the catheter to prevent the growth of bacteria/biofilms that accumulate on long-term catheters, percutaneous endoscopic gastronomy tubes (PEGS), peripherally inserted central catheter (PICC) lines, etc. (i.e., any long-term internal devices).

The catheter may prevent, eliminate, or remove any materials that can adhere to the catheter including but not limited to biofilms, bacteria, and salts. For example, uses of the catheter include at least one of preventing growth, preventing attachment, shaking off, or destroying bacteria, salts, and/or biofilms.

### Atherectomy

The catheter according to the present disclosure, or any other configuration of catheter including one or more EAP actuators, may be used to break down the buildup of medial calcification in the vasculature (e.g., pathological deposition of minerals in the vascular system) at any time during development, by operating the one or more EAP actuators at sonic frequencies/vibratory forces. The catheter according to the present disclosure may or example disrupt plaques as the plaques grow in the arteries, such that the plaques can be disrupted significantly earlier than current treatments allow.

### Tissue Effects

The catheter according to the present disclosure, or any other configuration of catheter, or any medical and/or therapeutic instrument, including one or more EAP actuators, may be used to produce desired tissue effects by the transmission of energy at one or more frequencies from the one or more EAP actuators. Transmission of energy at one or more frequencies from the one or more EAP actuators can increase nitric oxide production, impact vasodilation (e.g., cause increased vasodilation and/or reduced vasodilation depending on the one or more frequencies), increase adenosine triphosphate (ATP) production, reduce inflammatory response(s), and impact angiogenesis (e.g., increase or reduce). They may be used for example on any physiological lumen such as, but not limited to, an artery, vein, duct, ureters, etc.

### Sensing

The catheter according to the present disclosure, or any other configuration of catheter including one or more EAP actuators, optionally with a plurality of EAP actuators along the length of the shaft, may be used determine stenosed areas of a vessel the catheter is in based on changes in signals received (at the controller) from the one or more EAP actuators when the lumen becomes narrower (e.g., is pushed in by the stenosed area of the vessel), along with the ability to deliver localized therapeutic drugs to the atherectomy site through the open lumen.

The one or more EAP actuators may be further configured to detect wall contact. The one or more EAP actuators may be used as feedback or use with surgical robotics or for physician information. The one or more EAP actuators can be further configured to detect pressure changes. The one or more EAP actuators can be further configured to detect presence of thrombus at tip or inside the lumen of the catheter.

### Neurostimulation

The catheter according to the present disclosure, or any other configuration of catheter including one or more EAP actuators, may be used to provide stimulation of the nervous system. The catheter may provide mechanical stimulation via the vibrations of the one or more EAP actuators through a wall of a vein and/or artery to one or more nerves adjacent to or near the physiological lumen the catheter is inserted in (e.g., the vein and/or artery). Alternatively or additionally, the stimulation may be acoustic, ultrasonic, or the like that can be produced by the one or more EAP actuators.

### Low Friction Delivery

The catheter according to the present disclosure, or any other configuration of catheter including one or more EAP actuators, may be used as a delivery vehicle for stents, coils, implants, or other devices (e.g., delivered component) to reduce friction between the catheter wall and the delivered component is being delivered.

### Trackability

Friction between the catheter according to the present disclosure, or any other configuration of catheter including one or more EAP actuators and the vessel wall may be reduced due to the vibration of the one or more EAP actuators, resulting in easier and/or further navigation.

Actuator-induced deflection of the catheter shaft may be used to aid in steering (e.g., increase in controlled bendability). Actuator-induced opening of the distal tip may be used to increase diameter or create a funnel-like shape at the distal tip of the catheter, which can indicate where the distal tip is. The catheter according to the present disclosure, or any other configuration of catheter including one or more EAP actuators, may be used for detection purposes also—detection of locations of one or more portions of the catheter relative to the patient, detection of substances in the body (e.g., stenoses, cancers, blockages, or the like), or the like.

The actuators according to the present disclosure may be integrated in a balloon catheter.

In embodiments, a catheter may include the following features:
A flexible tube with a proximal tip, a distal tip, and a shaft extending from the proximal tip to the distal tip, where both distal and proximal tips have at least one opening;
One or more lumens for passing fluid (e.g., lytic agent, therapeutic agent, etc.) or guidewire through at least part of the catheter;
Expandable balloon on catheter (e.g., attached to at least a portion of the outside of the shaft of the catheter);
One or more EAP actuators integrated into the catheter shaft inside the balloon cavity and/or

One or more EAP actuators may be integrated into the balloon material, on the inside or outside surfaces, or integrated into the balloon film itself (as illustrated in FIGS. 8 and 9).

The balloon may be coated with one or more therapeutic agents on at least a portion of an exterior of the balloon.

The balloon may not be coated with one or more therapeutic agents and the balloon provides improved benefits to how energy from the one or more EAP actuators is delivered to lumen wall (e.g., stability, transfer of energy of any type, etc.).

The balloon catheter according to the present disclosure may be used to break down the buildup of medial calcification in the vasculature, using sonic frequencies/vibratory forces created by the one or more EAP actuators.

The balloon catheter according to the present disclosure (with or without drug coating) may be used for balloon angioplasty procedures to reopen the vessel for blood flow, reducing restenosis through any of the following mechanisms:
Increasing uptake of a drug, through vibration-induced opening of the endothelium, cavitation, and/or acoustic streaming
Enhanced vasodilation through endogenous production of NO
Softening and remodeling of the vessel tissue (specifically collagen)
Reduction in inflammatory response
Targeted delivery of drugs to angioplasty site through inner lumen (anesthetic, etc.)

U.S. patent application Ser. No. 18/638,401, filed Apr. 17, 2024, was incorporated herein in its entirety above. Any and all features described herein may be applied to any embodiment disclosed in the '401 Application, and any and all such modifications, alterations or combinations should be considered as disclosed in their entirety herein. The same likewise applies to the above-incorporated US 2022/0125454 A1.

The foregoing description is not intended to be limiting. To the contrary, the present application encompasses all variations, alterations modifications and/or equivalents within the spirit and scope of the appended claims.

1. A catheter device for the delivery of one or more therapeutic agents comprising:
a proximal end portion coupled to a controller configured to provide electrical signals; and a distal end portion comprising one or more electroactive polymer actuators configured for vibrational motion in response to the electrical signals;
wherein the catheter device is configured to carry the electrical signals from the controller to the one or more electroactive polymer actuators; and
wherein the catheter device is further configured to release one or more therapeutic agents from the distal end portion of the catheter device.

2. The catheter device according to claim 1, wherein the distal end portion of the device comprises one or more infusion ports, and the catheter device comprises at least one infusion lumen configured to carry the one or more therapeutic agents from the proximal end portion of the device to the one or more infusion ports.

3. The catheter device according to claim 1, wherein the distal end portion of the device is configured to release microbubbles.

4. The catheter device according to claim 1, wherein the device further comprises at least one occlusion device.

5.-8. (canceled)

9. A catheter device comprising:
a proximal end portion coupled to a controller configured to provide a electrical signal; and a distal end portion comprising one or more electroactive polymer actuators configured for vibrational motion in response to the electrical signal;
wherein the catheter device is configured to carry the electrical signal from the controller to the one or more electroactive polymer actuators; and
wherein the distal end portion of the catheter device further comprises a sleeve positioned over at least one of the one or more EAP actuators.

10. The catheter device according to claim 9, wherein the sleeve is mechanically decoupled from the one or more EAP actuators.

11. The catheter device according to claim 9, wherein the sleeve is an expandable sleeve.

12. The catheter device according to claim 9, wherein the one or more electroactive polymer actuators are located on an inner surface of the catheter wall, on an outer surface of the catheter wall, and/or are embedded in the catheter wall.

13. The catheter device according to claim 9, wherein the one or more electroactive polymer actuators are located on an inner surface of the sleeve and/or are embedded in a sleeve wall.

14. The catheter device according to claim 11, wherein the catheter device comprises at least one inflation lumen configured to expand the sleeve.

15. The catheter device according to claim 11, wherein the catheter device comprises one or more electroactive polymer actuators configured to transition the expandable sleeve between a first collapsed configuration and a second expanded configuration, in response to electrical signals provided by the controller.

16. The catheter device according to claim 9, wherein the catheter device is further configured to release one or more therapeutic agents.

17. The catheter device according to claim 16, wherein the sleeve comprises at least one coating comprising the one or more therapeutic agents.

18. A method for the delivery of a medical device into a patient, comprising the step of introducing the medical device in a lumen of a catheter device comprising:
a proximal end portion coupled to a controller configured to provide electrical signals; and a distal end portion comprising two or more electroactive polymer actuators configured for mechanical or vibrational motion in response to the electrical signals; wherein the electrical signals are provided in a predetermined coordinated pattern.

19.-34. (canceled)

35. The catheter device of claim 9, further comprising one or more conductors for carrying the electrical signal between the proximal end portion and the distal end portion, wherein the electroactive polymer actuator is electrically connected to said controller via said one or more conductors and wherein the electroactive polymer actuator expands and contracts in response to the application of the electrical signal.

36. The catheter device of claim 35, wherein the electroactive polymer actuator expands and contracts in response to the application of the electrical signal causing a strain response in a circumferential direction of the catheter.

37. The catheter device of claim 35, wherein said catheter is an aspiration catheter and has an opening at said distal end portion for ingestion of an occlusion or a portion thereof and wherein said shaft has a lumen for communicating pressure to said opening for ingestion of the occlusion or a portion thereof;
wherein the controller is adapted to generate said electrical signal to repeatedly expand and contract the electroactive polymer actuator in a vibrating manner for fracturing the occlusion for ingestion of the occlusion or the portion thereof through the opening.

38. The catheter device of claim 35, wherein the electroactive polymer actuator comprises two or more layers of electroactive polymer (EAP) films wrapped onto the distal end portion of the shaft,
wherein the EAP films are electrically connected to said controller via said one or more conductors and wherein the EAP films expand and contract in response to the application of the electrical signal.

39. The catheter device of claim 38, wherein the EAP films expand and contract in a circumferential direction of the catheter in response to the application of the electrical signal causing a strain response in the circumferential direction.

40. The catheter device according to claim 35, wherein the sleeve is attached to said distal end portion both distally and proximally of said at least one EAP actuator such that said sleeve is mechanically decoupled from said at least one EAP actuator and protects said at least one EAP actuator from liquid.

41. The catheter device according to claim 40, wherein the at least one EAP actuator is a single EAP actuator and wherein sleeve is attached to said distal end portion both distally and proximally of said single EAP actuator such that said sleeve is mechanically decoupled from said single EAP actuator and protects said single EAP actuator from liquid.

42. The catheter device according to claim 40 wherein the at least one EAP actuator is two or more EAP actuators and wherein sleeve is attached to said distal end portion both distally and proximally of said two or more EAP actuators such that said sleeve is mechanically decoupled from said two or more EAP actuators and protects said two or more EAP actuators from liquid.

43. The catheter device according to claim 38, wherein sleeve is attached to said distal end portion both distally and proximally of said at least one EAP actuator such that said sleeve is mechanically decoupled from said at least one EAP actuator and protects said at least one EAP actuator from liquid.

44. The catheter device according to claim 43, wherein the at least one EAP actuator is a single EAP actuator and wherein sleeve is attached to said distal end portion both distally and proximally of said single EAP actuator such that said sleeve is mechanically decoupled from said single EAP actuator and protects said single EAP actuator from liquid.

45. The catheter device according to claim 43, wherein the at least one EAP actuator is two or more EAP actuators and wherein sleeve is attached to said distal end portion both distally and proximally of said two or more EAP actuators such that said sleeve is mechanically decoupled from said two or more EAP actuators and protects said two or more EAP actuators from liquid.

46. The catheter device according to claim 35, wherein the sleeve is an expandable sleeve and wherein the catheter device further comprises at least one inflation lumen communicated to said sleeve for expanding the sleeve by inflation.

47. The catheter device according to claim 38, wherein the sleeve is an expandable sleeve and wherein the catheter device further comprises at least one inflation lumen communicated to said sleeve for expanding the sleeve by inflation.

48. The catheter device according to claim 40, wherein the sleeve is an expandable sleeve and wherein the catheter device further comprises at least one inflation lumen communicated to said sleeve for expanding the sleeve by inflation.

49. The catheter device according to claim 43, wherein the sleeve is an expandable sleeve and wherein the catheter device further comprises at least one inflation lumen communicated to said sleeve for expanding the sleeve by inflation.
